(19) United States
(12) Patent Application Publication
(10) Pub. No.: US 2023/0120909 A1
(43) Pub. Date: Apr. 20, 2023

(54) BREATH SENSING DEVICE

(71) Applicant: PI BIOELECTRONICS COMPANY LIMITED, TAICHUNG (TW)

(72) Inventors: Min-Hui CHIOUCHANG, TAICHUNG (TW); Wei-Ting HSIEH, TAICHUNG (TW); Yung-Jiun LIN, TAICHUNG (TW)

(21) Appl. No.: 17/502,809

(22) Filed: Oct. 15, 2021

(51) Int. Cl. A61B 5/091; A61B 5/00

(52) U.S. Cl. CPC A61B 5/091; A61B 5/6892; A61B 5/7221; A61B 2562/0247; A61B 2562/0271; A61B 2562/0238; A61B 2562/046

(57) ABSTRACT

The invention is a breath sensing device comprising a sensing pad, a plurality of coverage sensing units, and a signal processor, the coverage sensing units are disposed on the sensing pad, when a user lies down on the sensing pad, a plurality of coverage signals sensed by the coverage sensing units respectively produce a periodic change according to the deformation of a curve along the body of the user while breathing, the signal processor is electrically connected to the coverage sensing units to receive the coverage signals, and the signal processor calculates to obtain duration and proportion of the user's inspiration, expiration, and pause phase and depth information of breathing from a waveform of the coverage signals based on a signal-processing algorithm.

### FIELD OF THE INVENTION

The invention is related to breath sensing, and more particularly to a breath sensing device.

### BACKGROUND OF THE INVENTION

Respiration or breathing is an important sign of human life. For lung infections and pulmonary infiltrations caused by viruses (such as Coronavirus disease, COVID-19) in long-term bedridden patients, middle-aged and elderly people with chronic diseases such as hypertension and hyperlipidemia, long-term monitoring of breathing detects changes in the patient's condition and take preventive treatment, or in case of emergency, take emergency treatment as soon as possible. The breathing process comprises: quick inspiratory phase, slight slowdown, short inspiratory pause, long expiratory phase, long expiratory pause, and followed by the next breath. In clinical evaluation of breathing condition, in addition to directly observing the upward and downward movements of the chest, it also evaluates the speed of breathing (the normal duration of inspiration, expiration, and pause phase is about 1, 1.5, and 1 in seconds respectively); depth (roughly evaluated based on the degree of chest expansion and the amount of airflow in and out of the respiratory tract, which cannot be quantified); rhythm (regular and smooth); and difficulty level. Diagnoses are based on the clinical evaluations thereof, for example, whether the person being evaluated has tachypnea (the breathing depth is normal but the breathing frequency is more than 24 times per minute); oligopnea (the breathing depth is normal but the breathing frequency is less than 8 times per minute); hyperpnea (minute ventilation increases when the breathing depth increases and the breathing frequency is normal); hypopnea (the breathing frequency is normal but the breathing depth is shallower); hyperventilation (hyperpnea and tachypnea); hypoventilation (oligopnea and hypopnea, particularly with a prolonged rest period); or other special cases of breathing (continuous changes in breathing depth or long pauses in breathing)

In the conventional method of sensing breath, as provided in U.S. Pat. No. 6,478,747, “Method and device to sense breathing”, the user has to wear a breath sensor on the nasal cavity to directly sense the airflow of the user's breath. However, it is suitable only for emergency patients. Because the respirator will insert into the user's nose and mouth, after long-term use, in addition to the discomfort feeling in the user's nose and mouth, long-term fixation at the mouth and nose often leads to pressure injuries (bedsores) on the user's face and increase the risk of infection.

In another example, the U.S. Pat. No. 8,663,126 provides the “Wearable acoustic device for monitoring breathing sounds”, which is a wearable breath sensor that allows the user to wear directly on the chest to sense the chest expansion during breathing in order to sense the user's breathing condition. Compared with the mouth-nose type breath sensor, it is more suitable for patients with chronic diseases to use at home. However, this method requires the user to tie the device on the chest, which makes the user feel limited and uncomfortable, and affects the quality of sleep. Long-term wear may also cause pressure injuries (bedsores) to the user.

In another example, the U.S. Pat. No. 11,013,415 provides the

“Hydraulic bed sensor and system for non-invasive monitoring of physiological data”, which is a hydraulic bed sensor that transmits vibration through a liquid. The sensor includes two parts, which are the liquid vibration transmission module for receiving the user's heartbeat and breath vibration, and the pressure sensing module for measuring the pressure change caused by vibration in the liquid vibration transmission module. The liquid vibration transmission module installed under the mattress to sense the vibration signals in response to the heartbeat and breath vibration of the user lying on the mattress in a non-invasive manner. Through analysis and processing of the measured vibration signals, the user's heartbeat and breathing frequencies are obtained. The user does not need to wear any instrument, and there is no problem with affecting the quality of sleep.

However, the hydraulic bed sensor only collects the vibration signals generated by the user's heartbeat and breathing through the liquid vibration transmission module; in fact, the module not only receives the vibration signals generated by the user's heartbeat and breathing, but the vibration signals are also mixed with various vibration noises in the environment that belong to the frequency range of human breathing and heartbeat. After using a cumbersome signal processing technique to analyze the frequencies of all the vibration signals, it is also required to retrieve the user's heartbeat and breathing frequencies. Therefore, the user's heartbeat and breathing frequencies are easily interfered and distorted by the various vibration noises that belong to the frequency range of human breathing and heartbeat. Although the hydraulic bed sensor has the advantage of no need for the patient to wear the sensor when estimating the breathing frequency, it lacks the speed and depth information of ventilation and thus cannot be applied in evaluating breathing

### SUMMARY OF THE INVENTION

A main object of the invention is to provide a breath sensing device that does not need to be worn, measures the duration of inspiration, expiration, and pause phase, respectively, measures depth information of breathing, avoids measurement bias by eliminating noises from vibration in an external environment, and simplifies the signal analysis process.

In order to achieve the above object, the invention is a breath sensing device provided on or inside a mattress on which a user is lying, to sense a change in a coverage area of the mattress covered by the user. The change in the coverage area is in response to a volume change of a body cavity when the user breathes. The volume change of the body cavity directly reflects speed and depth of breathing, and therefore, detecting the volume change of the body cavity is equivalent to detecting speed and depth information of breathing. Duration and proportion of inspiration, expiration, and pause phase, as well as breathing frequency, are obtained through signal processing. The breath sensing device comprises a sensing pad, a plurality of coverage sensing units, and a signal processor, wherein the sensing pad comprises a surface layer and a supporting pad body, the surface layer is provided for the user to lie thereon, and the supporting pad body is located under the surface layer. The plurality of coverage sensing units are disposed on the supporting pad body. Each of the plurality of coverage sensing units senses the change in the coverage area and generates a plurality of coverage signals, when the user lies on the surface layer of the sensing pad. The plurality of coverage signals respectively generated by parts of the plurality of coverage sensing units perform a periodic change according to the deformation of a curve along the body of the user while breathing. Furthermore, there are three types of the coverage area according to the curve along the body of the user on the surface layer. The first type of the coverage area is located next to the user and will not be covered by the user throughout the breathing process. The second type is located directly under the user and will be totally covered by the user throughout the breathing process. The third type is located between the first type and the second type and will perform the periodic change according to the curve along the body of the user while breathing. The signal processor is electrically connected to the plurality of coverage sensing units to receive the plurality of coverage signals. The signal processor retrieves the plurality of coverage signals with the periodic change based on a signal-processing algorithm, selecting one coverage signal which comprises a best signal quality from the plurality of coverage signals with the periodic change as a basis, and analyzing a waveform of the basis to obtain breathing depth information, duration and proportion of time of the user's inspiration, expiration, and pause phase.

As mentioned above, in the breath sensing device of the invention, the user does not need to wear an instrument, simply lying on the sensing pad is capable of measuring breathing depth information, the duration and proportion of the user's inspiration, expiration, and pause phase, in order to obtain the breathing frequency. The invention measures the periodic change according to the deformation of the curve along the body of the user while breathing, without the interference of noises from external environmental vibrations, and therefore is capable of simplifying the signal analysis process and avoiding measurement bias.

### BRIEF DESCRIPTION OF THE DRAWINGS

FIG. 1 is a top view of a breath sensing device according to the invention;

FIG. 2 is a schematic diagram of a coronal plane of a user and the invention;

FIG. 3A is a schematic diagram of measuring the user's breathing according to the invention;

FIG. 3B is a schematic diagram of deformation of a curve along the body of the user and the invention;

FIG. 4 is a line graph of a largest periodic change according to the invention;

FIG. 5 is a top view of another embodiment of the breath sensing device according to the invention;

FIG. 6 is a cross-sectional view of still another embodiment of the breath sensing device according to the invention; and

FIG. 7 is a schematic diagram of a plurality of coverage sensing units according to the invention.

### DETAILED DESCRIPTION OF THE PREFERRED EMBODIMENTS

The detailed description and technical contents of the invention are described below with reference to the drawings.

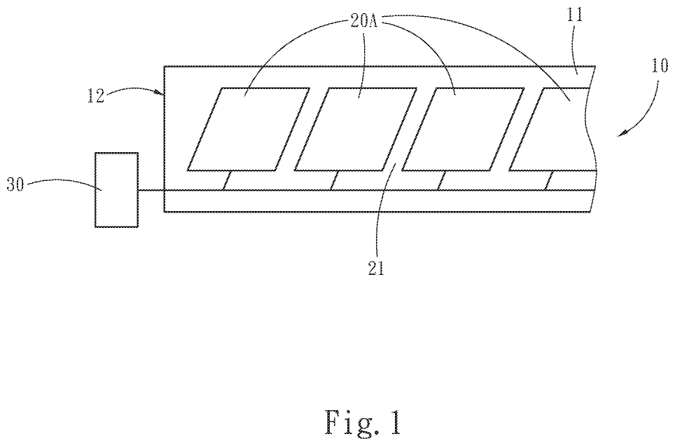

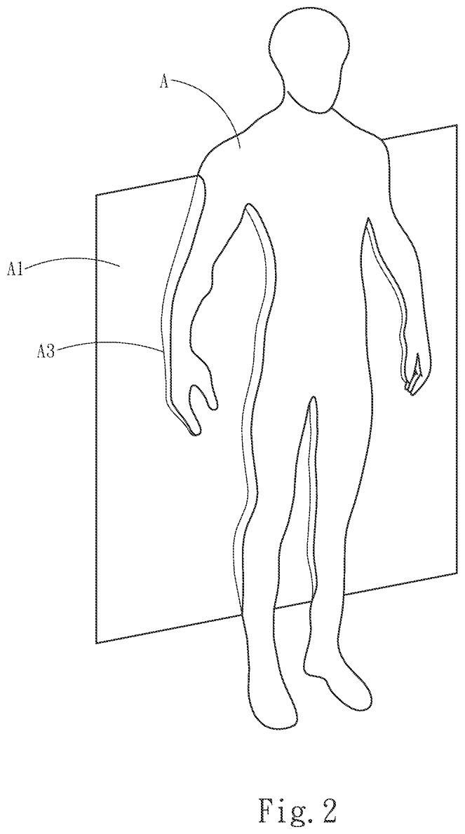

Please refer to FIG. 1 and FIG. 2. The invention is a breath sensing device to sense the speed and depth information of breathing of a user A. The breath sensing device comprises a sensing pad 10, a plurality of coverage sensing units 20A, and a signal processor 30. The sensing pad 10 comprises a surface layer 11 and a supporting pad body 12, wherein the surface layer 11 is provided for the user A to lie thereon, and the supporting pad body 12 is located under the surface layer 11. The plurality of coverage sensing units 20A are disposed on the supporting pad body 12. In an embodiment, the plurality of coverage sensing units 20A are hidden in the supporting pad body 12, and preferably, a gap 21 is disposed between any two adjacent coverage sensing units 20A. The plurality of coverage sensing units 20A are selected from a group of contact sensors consisting of capacitive pressure sensors, resistive pressure sensors, optical pressure sensors, and temperature sensors; or selected from a group of non-contact sensors consisting of photo-interrupt sensor, capacitive proximity sensor, Hall element sensor, and optical temperature sensor. Accordingly, the plurality of coverage sensing units 20A sense a coverage rate of the surface layer 11 to generate a plurality of coverage signals.

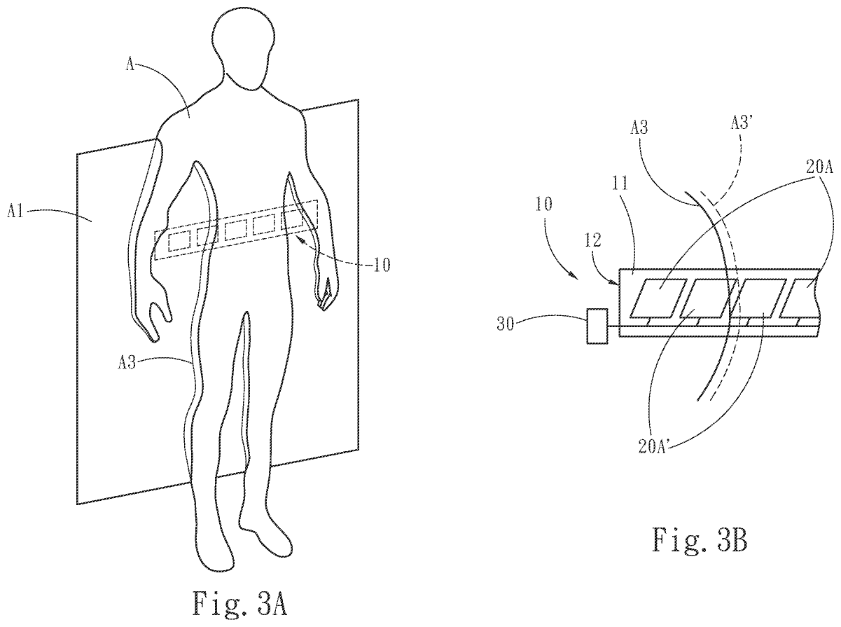

Please refer to FIG. 3A and FIG. 3B. An intersection of the user A and a coronal plane A1 is a curve A3. When the user A is breathing, the curve A3 of the user A will be deformed in response to the breathing and transformed into a curve A3′. Areas where the curve A3 deforms significantly are the thorax and the abdomen. Therefore, when the user A lies on the surface layer 11 of the sensing pad 10, the plurality of coverage sensing units 20A generate the plurality of coverage signals, and the plurality of coverage sensing units 20A can be divided into four types as follow. First type: parts of the plurality of coverage sensing units 20A is completely covered, and respectively output a maximum value. Second type: parts of the plurality of coverage sensing units 20A are not covered, and respectively output a value of “0”, in other words, the second type of the coverage sensing units 20A are entirely uncovered. Third type: parts of the plurality of coverage sensing units 20A are covered by body parts, such as arms, of the user A that do not deform the curve of the body while breathing, and respectively output a fixed value. Fourth type: as shown in FIG. FIG. 3B, parts of the plurality of coverage sensing units 20A′ located at edges of the body respectively output a periodic value according to a periodic coverage change caused by the curve A3 being deformed into the curve A3′ while the user A breathing

In an embodiment, the plurality of coverage sensing units 20A are designed as diamond or non-linear shapes, so that the adjacent coverage sensing units 20A overlap with each other on a plane. As shown in FIG. 3B, the gap 21 is disposed between the two adjacent coverage sensing units 20A, and the gap 21 is not parallel to the body curve of the user A. Accordingly, when the user A breathes, the curve A3 is deformed into the curve A3′, and if the deformation is smaller than the gap 21 and is located between the two adjacent coverage sensing units 20A′, there will be at least one of the coverage sensing units 20A′ that is capable of detecting a coverage signal representing a coverage change.

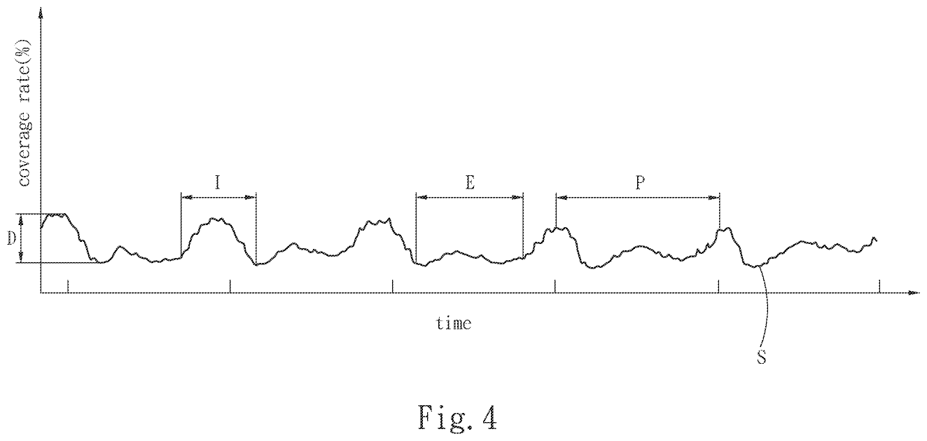

Please refer to FIG. 4 as well. The signal processor 30 is electrically connected to the plurality of coverage sensing units 20A to receive the plurality of coverage signals. The signal processor 30 retrieves the plurality of coverage signals with a periodic change based on a signal-processing algorithm, selecting one coverage signal which comprises a best signal quality from the plurality of coverage signals with the periodic change as a basis, and analyzing a waveform of the basis to obtain breathing depth information, duration and proportion of time of the user's inspiration, expiration, and pause phase. Further, the signal processor 30 analyzes an inspiratory duration I, an expiratory duration E, a breathing depth D, and a period P of one coverage signal S with the largest value change, and converts the above values into a breathing frequency of the user A.

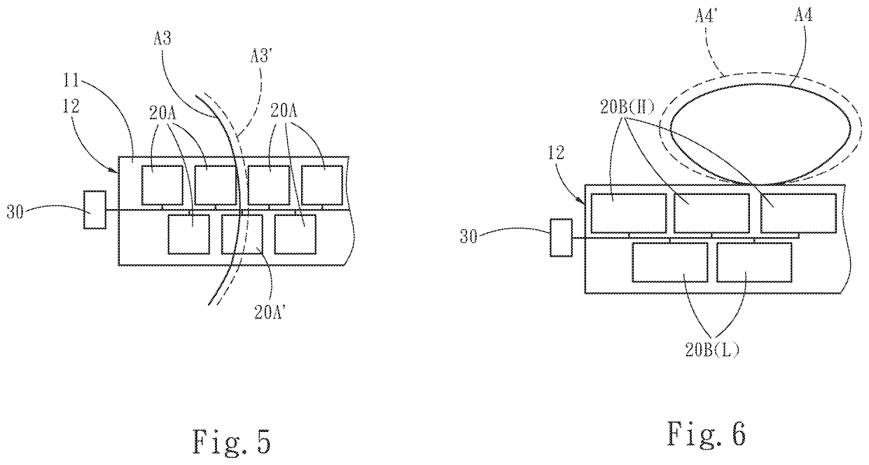

Please refer to FIG. 5 for another implementation structure of the breath sensing device of the invention, wherein the plurality of coverage sensing units 20A are arranged in an array. In this arrangement, when the curve A3 of the user A is deformed into the curve A3′ while the user A breathing, the plurality of coverage sensing unit 20A′ located at edges of the body respectively output a periodic value according to the periodic coverage change.

Please refer to FIG. 6 for a cross-sectional view of another embodiment of the breath sensing device of the invention. A plurality of coverage sensing units 20B are arranged as a multi-layered overlapping structure, and are divided into an upper layer set H and a lower layer set L. The plurality of coverage sensing units 20B of the upper layer set H overlap vertically with the plurality of coverage sensing units 20B of the lower layer set L.

In one embodiment, the plurality of coverage sensing units 20B of the upper layer set H and the plurality of coverage sensing units 20B of the lower layer set L are vertically staggered and stacked. In this configuration, planes bounded by thorax A4, A4′ of the user A are changed while the user A breathing, causing a coverage rate of the surface layer 11 being changed. The plurality of coverage sensing units 20B are capable of sensing the coverage rate of the surface layer 11 to generate the plurality of coverage signals.

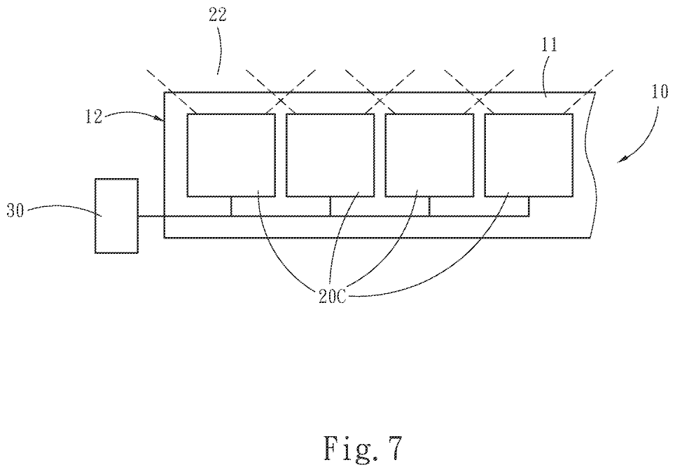

Please refer to FIG. 7 for a schematic diagram of an implementation structure of the plurality of coverage sensing units 20C of the breath sensing device. The plurality of coverage sensing units 20C are selected from a group of non-contact sensors consisting of photo-interrupt sensor, capacitive proximity sensor, Hall element sensor, and optical temperature sensor. In this embodiment, each of the plurality of coverage sensing units 20C comprises a sensing area 22 formed on the surface layer 11, and the sensing area 22 has a wide-angle sensing range, so that the sensing areas 22 of the plurality of coverage sensing units 20C formed on the surface layer 11 overlap with one another without blind zone. In this embodiment, the plurality of coverage sensing units 20C are capable of respectively sensing the coverage rate of the surface layer 11 being covered to generate the coverage signals.

Accordingly, comparing with the prior art, the invention comprises the following advantages:

1. The ventilation of the user can be measured once the user simply lying on the sensing pad, whether lying face up, lying sideway or lying face down, and the user does not need to wear any instrument.

2. The invention measures the curve deformation of the user's body while breathing to obtain the periodic value, avoiding the problem of the noise interference caused by the external environment, and therefore the invention is capable of simplifying the signal analysis process and reducing measurement bias.

3. The invention is not only capable of measuring the breathing frequency, but also capable of measuring the duration, speed and depth information of the ventilation for effective evaluation of breathing clinically.

What is claimed is:

1. A breath sensing device, provided on a mattress on which a user is lying to sense a change in a coverage area of the mattress covered by the user, comprising:
a sensing pad, comprising a surface layer provided for the user to lie thereon and a supporting pad body located under the surface layer;
a plurality of coverage sensing units, disposed on the supporting pad body, each of the plurality of coverage sensing units sensing the change in the coverage area and generating a plurality of coverage signals, wherein the plurality of coverage signals generated by parts of the plurality of coverage sensing units performs a periodic change according to the deformation of a curve along a body of the user while breathing; and
a signal processor, electrically connected to the plurality of coverage sensing units to receive the plurality of coverage signals, the signal processor retrieving the plurality of coverage signals with the periodic change based on a signal-processing algorithm, selecting one coverage signal which comprises a best signal quality from the plurality of coverage signals with the periodic change as a basis, and analyzing a waveform of the basis to obtain breathing depth information, duration and proportion of time of the user's inspiration, expiration, and pause phase.

2. The breath sensing device as claimed in claim 1, wherein the plurality of coverage sensing units are contact sensors and are selected from the group consisting of capacitive pressure sensors, resistive pressure sensors, optical pressure sensors, and temperature sensors.

3. The breath sensing device as claimed in claim 1, wherein a gap is disposed between two adjacent coverage sensing units, and the gap is not parallel to the curve along the body of the user.

4. The breath sensing device as claimed in claim 1, wherein the plurality of coverage sensing units are arranged in an array.

5. The breath sensing device as claimed in claim 1, wherein the plurality of coverage sensing units are divided into an upper layer set and a lower layer set according to disposing height, and the plurality of coverage sensing units of the upper layer set and the plurality of coverage sensing units of the lower layer set are vertically staggered and stacked.

6. The breath sensing device as claimed in claim 1, wherein the plurality of coverage sensing units are non-contact sensors and are selected from the group consisting of photo-interrupt sensor, capacitive proximity sensor, Hall element sensor, and optical temperature sensor.

7. The breath sensing device as claimed in claim 6, wherein the plurality of coverage sensing units comprise a plurality of sensing areas formed on the surface layer respectively, and the plurality of sensing areas overlap with one another.
